(19) United States
(12) Patent Application Publication
(10) Pub. No.: US 2024/0325279 A1
(43) Pub. Date: Oct. 3, 2024

(54) COLOR RETENTION SYSTEMS USING DENDRIMER AND CHITOSAN COMPLEX FOR LONG-LASTING EFFECT

(71) Applicant: Henkel AG & Co. KGaA, Duesseldorf (DE)

(72) Inventors: Gerhard Frederick Kummerow Casas, Milford, CT (US); Jennifer S. Haghpanah, New York, NY (US); Andrew Savaides, Norwalk, CT (US); Jeffrey S. Bankers, Stamford, CT (US)

(21) Appl. No.: 18/741,918

(22) Filed: Jun. 13, 2024

Related U.S. Application Data
(63) Continuation of application No. PCT/US2022/052819, filed on Dec. 14, 2022.
(60) Provisional application No. 63/289,494, filed on Dec. 14, 2021.

(51) Int. Cl. A61K 8/73; A61K 8/33; A61K 8/81; A61K 8/88; A61Q 5/00; A61Q 5/02

(52) U.S. Cl. CPC A61K 8/736; A61K 8/33; A61K 8/73; A61K 8/8111; A61K 8/88; A61Q 5/004; A61Q 5/02

(57) ABSTRACT

A hair color retention system includes a primer composition having an effective amount of a dendrimer or hyperbranched compound in a cosmetically acceptable aqueous carrier and a chitosan complex composition including chitosan, dehydroxanthan gum, an aldehyde-bearing compound (ABC), and an organic acid. Upon application and rinsing of the primer composition, followed by application of the chitosan complex composition, a chitosan complex coating is formed on hair fibers that provides color retention to the hair. ΔE of the treated hair is less than or equal to 10 after 40 washes with a clarifying shampoo.

### CROSS-REFERENCE TO RELATED APPLICATIONS

This application is a continuation application of International Application No. PCT/US2022/052819, filed Dec. 14, 2022, which claims benefit of U.S. Provisional Application No. 63/289,494, filed on Dec. 14, 2021, both of which are incorporated by reference herein in their entirety for all purposes.

### FIELD OF THE INVENTION

The invention relates to a hair care system including a primer composition comprising a dendrimer or a hyperbranched compound and a chitosan complex applied after or over the primer composition. The system is suited for retaining or minimizing color change of the color of color-treated hair treated therewith.

### BACKGROUND OF THE INVENTION

In practice, dye films deposited on the hair are susceptible to extraction by repeated shampooing, erosion by combing and brushing, and fading by exposure to sunlight and oxygen. Red colors are particularly susceptible to these degrading processes.

An object of the invention is to provide hair care compositions and methods that will provide resistance to erosion, abrasion, and chemical fading of the color. A still further objective is to provide a convenient kit that can be used by individual consumers to practice the method.

Chitosan compositions are known for hair care but typically are used to smooth, condition or “fix” the hair by forming a film on the hair. For example, U.S. Patent Application Publication No. 2017/0360671 describes chitosan compositions including an aldehyde-bearing compound, and an organic acid that can form a semi-permanent film on a hair fiber in the presence of heat.

There is a desire to improve known chitosan hair compositions so that they provide color retention for color-treated hair or other fibers.

### SUMMARY OF THE INVENTION

Surprisingly, it has been found that a chitosan complex can provide long lasting color retention of color-treated hair when applied over a primer composition comprising a dendrimer or hyperbranched compound.

Thus, in one aspect, the invention relates to a treatment system for retaining the color of color-treated fibers comprising a primer composition comprising an effective amount of a dendrimer or hyperbranched compound in a cosmetically acceptable aqueous carrier, and a chitosan complex composition comprising chitosan, dehydroxanthan gum, an aldehyde-bearing compound (ABC), and an organic. Upon sequential application of the primer composition and the chitosan complex composition, a composition is formed that provides color retention to the color-treated fibers.

In certain embodiments, the primer composition comprises about 0.005 to about 5% by weight of the dendrimer or hyperbranched compound, more preferably about 0.01 to about 3.5% by weight, most preferably about 0.02 to about 3% by weight of the primer composition.

In some embodiments, the dendrimer or hyperbranched compound is selected from the group consisting of polylysines, amylopectins, poly alpha-olefins, organopolysiloxane copolymers, polyquaterniums, and combinations thereof. In some of those embodiments, the compound is poly-L-lysine, or Hydrogenated Poly(C6-14 Olefin).

In certain embodiments, the aldehyde-bearing compound (ABC) is selected from the group consisting of vanillin, p-vanillin, o-vanillin, or ethylvanillin.

In some embodiments, the organic acid is selected from the group consisting of acid is selected from the group consisting of acetic acid, formic acid, glycolic acid, lactic acid, ascorbic acid, pyruvic acid, salicylic acid, citric acid, adipic acid, succinic acid, malic acid, tartaric acid, fumaric acid, glyoxilic acid, and combinations thereof. Preferably, the organic acid is acetic acid, most preferably glacial acetic acid.

In certain embodiments, the chitosan comprises about 0.2 to about 3.0% by weight of the chitosan complex composition, more preferably 0.4 to 1.5%, most preferably 0.5 to 1.0. % by weight.

Preferably, dehydroxanthan gum comprises about 0.1 to about 3% by weight of the chitosan complex composition, more preferably 0.2 to 2.0% by weight, most preferably 0.5 to 1.5. % by weight.

In certain preferred embodiments, the fiber is hair.

In some preferred embodiments, the primer composition is a shampoo comprising an effective amount of a hyperbranched or dendrimer compound in a cosmetically acceptable aqueous carrier. In certain of those embodiments, the shampoo further comprises about 5 to about 30% by weight of total active surfactant, more preferably about 10 to about 20% by weight, most preferably about 12 to about 15% by weight. In certain of those embodiments, the surfactant composition comprises a surfactant selected from the group consisting of lauryl glucoside, cocoamidopropyl betaine, sodium lauroyl methyl isethionate, sodium methyl 2-sulfolaurate, sodium methyl cocoyl taurate, and combinations thereof.

As used herein, “shampoo” refers to a cleansing composition for keratinous fibres, particularly human hair.

Accordingly, in a second aspect, the invention provides a color retention system for color-treated hair comprising a shampoo composition comprising an effective amount of a dendrimer or hyperbranched compound in a cosmetically acceptable aqueous carrier; and a chitosan complex composition comprising chitosan, dehydroxanthan gum, an aldehyde-bearing compound (ABC), and an organic acid. Upon application and rinsing of the shampoo followed by application of the chitosan complex composition a composition is formed that provides color retention to the color-treated hair.

The term “effective amount” means generally at least a concentration of at least 0.001% by weight based on the weight of the total composition. Preferably, a concentration of 0.005-5 wt. %, more preferred in the range of 0.01 to 3 wt.-% is used. In certain exemplary embodiments, about 0.04% to about 2.5% of a hyperbranched dendrimer is contained in a cosmetically acceptable aqueous carrier.

In some embodiments, the hyperbranched or dendrimer compound in the shampoo is selected from the group consisting of polylysines, amylopectins, poly alpha-olefins, organopolysiloxane copolymers, polyquaterniums, and combinations thereof. In some preferred embodiments, the dendrimer or hyperbranched compound is a dendrimer formed from a mixture of glycerin, water, and polylysine, or a polyalphaolefin. In certain particularly preferred embodiments, the hyperbranched or dendrimer compound is poly-L-lysine or Hydrogenated Poly(C6-14 Olefin).

In certain embodiments, the dendrimer or hyperbranched compound comprises about 0.005 to about 5% by weight of the shampoo composition, more preferably about 0.01 to about 3.5% by weight, more preferably about 0.02 to about 3% by weight of the shampoo composition.

In some embodiments, the shampoo further comprises about 5 to about 30% by weight of active surfactant in a surfactant composition, more preferably about 10 to about 20% by weight. Most preferably, the primer/shampoo comprises about 12 to about 15% by weight active surfactant.

In certain embodiments, the shampoo composition comprises a nonionic surfactant, preferably the nonionic surfactant comprises, consists essentially of, or consists of a mono-alkyl glucoside, such as lauryl glucoside and/or caprylyl/capryl glucoside.

In some embodiments, the shampoo contains one or more co-surfactants. In certain embodiments, the co-surfactants are selected from the group consisting of cocamidopropyl betaine, sodium lauroyl methyl isethionate, sodium methyl 2-sulfolaurate, sodium methyl cocoyl taurate, sodium methyl oleoyl taurate, and combinations thereof.

In certain embodiments, the surfactant composition comprises a surfactant selected from the group consisting of lauryl glucoside, cocoamidopropyl betaine, sodium lauroyl methyl isethionate, sodium methyl 2-sulfolaurate, sodium methyl cocoyl taurate, and combinations thereof.

Preferably, the shampoo is sulfate free, for example, does not contain anionic sulfate surfactants such as sodium lauryl sulfate.

In some embodiments, the aldehyde-bearing compound (ABC) is selected from the group consisting of vanillin, p-vanillin, o-vanillin, or ethylvanillin. In some of those embodiments, the ABC is vanillin.

In certain embodiments, the organic acid is selected from the group consisting of acetic acid, formic acid, glycolic acid, lactic acid, ascorbic acid, pyruvic acid, salicylic acid, citric acid, adipic acid, succinic acid, malic acid, tartaric acid, fumaric acid, glyoxilic acid, and combinations thereof. Preferably, the organic acid is acetic acid, most preferably glacial acetic acid.

In some embodiments, the chitosan comprises about 0.3 to about 3% by weight of the chitosan complex composition, more preferably 0.4 to 1.5%, most preferably 0.5 to 1.0% by weight of the complex.

In certain embodiments, the dehydroxanthan gum comprises about 0.1 to about 3% by weight of the chitosan complex composition, more preferably 0.2 to 2.0%, most preferably 0.5 to 1.5% by weight of the complex.

In another aspect, a composition for use in retaining color on a fiber comprises a homogeneous dispersion of chitosan, dehydroxanthan gum, vanillin, and glacial acetic acid. The chitosan comprises about 0.5 to about 1.0% by weight of the composition, and the dehydroxanthan gum comprises about 0.25 to about 2.0% by weight of the composition. The composition comprises a soft complex and is free of brittle precipitates.

Thus, in yet another aspect, the invention provides a method of maintaining initial color of color-treated hair comprising topically applying a shampoo composition comprising a dendrimer or hyperbranched compound to color-treated hair, rinsing the shampoo from the hair, topically applying an effective amount of a chitosan complex composition comprising a chitosan, dehydroxanthan gum, an aldehyde-bearing compound, and an organic acid to the rinsed hair, and subsequently drying the hair so that the chitosan complex composition forms a crosslinked complex with the hair. Advantageously, the crosslinked complex is maintained on and minimizes change of the color of the hair for 10 washes with a clarifying shampoo, preferably for 20 washes, more preferably for 30 washes, most preferably for 40 washes.

In some embodiments, the step of drying comprises blowing the hair with air at temperature of about 50° C. or greater for about 2 minutes.

In certain embodiments, the step of drying includes brushing the hair while applying heated air with a blow dryer.

In some embodiments, the shampoo comprises about 0.005 to about 5% of the dendrimer or hyperbranched compound.

Preferably, the shampoo is free of sulfates.

Preferably, the chitosan complex composition is free of precipitates.

In certain embodiments, the chitosan complex composition comprises about 0.5 to about 1.0% by weight chitosan, and about 0.25 to about 2.0% by weight dehydroxanthan gum.

In some embodiments, the dendrimer or hyperbranched compound is selected from the group consisting of polylysines, amylopectins, poly alpha-olefins, organopolysiloxane copolymers, polyquaterniums, and combinations thereof. In certain of those embodiments, the dendrimer or hyperbranched compound is poly-L-lysine, or Hydrogenated Poly(C6-14 Olefin).

In certain embodiments, ΔE of the dried hair is less than or equal to 10 after 40 washes with a clarifying shampoo. Preferably ΔE is less than 8, more preferably less than 6, most preferably less than 4 after 40 washes with a clarifying shampoo.

The invention also provides a method of reducing color change of a dyed fiber comprising topically applying an effective amount of a dendrimer or hyperbranched compound to the fiber, topically applying an effective amount of a chitosan complex composition comprising a chitosan, dehydroxanthan gum, an aldehyde-bearing compound, and an organic acid to the fiber on which the dendrimer or hyperbranched compound has been applied, and exposing the topically treated fiber to a temperature of about 50° C. or greater for about 2 minutes. In certain embodiments, ΔE of the fiber is less than 10 after 40 washes with a clarifying shampoo. Preferably ΔE is less than 8, more preferably less than 6, most preferably less than 4 after 40 washes with a clarifying shampoo.

In some embodiments, the dendrimer or hyperbranched compound is selected from the group consisting of polylysines, amylopectins, poly alpha-olefins, organopolysiloxane copolymers, polyquaterniums, and combinations thereof. In some of those embodiments, the dendrimer or hyperbranched compound is poly-L-lysine, or Hydrogenated Poly(C6-14 Olefin).

In certain embodiments, the dendrimer or hyperbranched compound is formulated in an aqueous carrier composition at about 0.5 to about 5% by weight of the composition. In some of those embodiments, the aqueous carrier composition comprises total active surfactant in an amount of about 5 to about 30% by weight of the composition comprising the dendrimer or hyperbranched compound and aqueous carrier.

In some embodiments, the chitosan complex composition is a homogeneous dispersion free of precipitates, the chitosan comprises about 0.5 to about 1.0% by weight of the chitosan composition, and the dehydroxanthan gum comprises about 0.25 to about 2.0% by weight of the chitosan composition.

In certain embodiments, the aldehyde-bearing compound (ABC) is selected from the group consisting of vanillin, p-vanillin, o-vanillin, or ethylvanillin.

In some embodiments, the organic acid is selected from the group consisting of acid is selected from the group consisting of acetic acid, formic acid, glycolic acid, lactic acid, ascorbic acid, pyruvic acid, salicylic acid, citric acid, adipic acid, succinic acid, malic acid, tartaric acid, fumaric acid, glyoxilic acid, and combinations thereof.

Also provides is a kit for retaining initial color of color-treated hair comprising a first container comprising a primer composition, the primer composition comprising a dendrimer or hyperbranched compound, a second container comprising a chitosan complex composition, the chitosan complex composition comprising chitosan, an aldehyde-bearing compound, dehydroxanthan gum, and an organic acid, and instructions indicating a user should apply the primer composition to color-treated hair followed by application of the chitosan complex composition.

### BRIEF DESCRIPTION OF THE DRAWINGS

FIG. 1 shows FTIR spectra of chitosan HD (top) and a chitosan HD complex of Example 1 (bottom).

FIG. 2A is a photograph showing a cast chitosan HD film, and a chitosan HD-complex film.

FIG. 2B is a photograph showing a chitosan HD complex+dendrimer film in a beaker of water containing 1% shampoo.

FIG. 2C is a schematic depicting proposed reaction for the formation of a chitosan complex.

FIG. 3 is a photograph showing various mannequin heads for which the hair has been treated with various compositions described in Examples 2 and 4 followed by 10 shampoos with a stripping shampoo and blow dry with a round brush. The left side of each mannequin shows results for compositions of Example 2. The right side of each mannequin shows results for application of compositions of Example 2 followed by application of the Chitosan Serum of Example 4.

FIG. 4 is a bar graph of (a) values from Red80 experiments on single bleached hair swatches in accordance with Example 6 wherein the swatches have been treated with various compositions of Examples 2 and 4 and thereafter shampooed 10 times with a stripping shampoo.

FIG. 5 is a bar graph of (a) values from Red80 experiments on platinum bleached hair swatches in accordance with Example 6 wherein the swatches have been treated with various compositions of Examples 2 and 4 and thereafter shampooed 10 times with a stripping shampoo.

FIG. 6 is a bar graph of volume measurements (cm) on virgin, single bleached, and platinum bleached hair swatches from experiments in accordance with Example 7 wherein the swatches have been treated with various compositions of Examples 2 and 4 and thereafter shampooed 10 times with a stripping shampoo.

FIG. 7 is a bar graph of the change in color ΔE of red dyed hair swatches from experiments in accordance with Example 8 wherein the swatches have been treated with various compositions of Examples 2 and 4 and thereafter shampooed 40 times with a stripping shampoo

### DETAILED DESCRIPTION OF THE INVENTION

The invention described herein includes methods and compositions that, when practiced, form a color retention composition on the hair or other fibers. The color retention composition is capable of supplying certain enhanced benefits to human hair and/or other types of fibers, including increased volume, shine, and luster, and smoothness. The color retention compositions are comprised of a dendrimer or hyperbranched compound composition, preferably in an aqueous-based shampoo carrier that can be used to prime the dendrimer or hyperbranched compound onto a fiber substrate, and a chitosan complex composition applied over the hyperbranched dendrimer.

In the practice of the method, a system for retaining the color of color-treated hair includes topically applying to a fiber a dendrimer or hyperbranched compound containing primer composition followed by applying a chitosan complex to the dendrimer-primed fiber. The compositions may be applied by any means in the art, including, for example, combing, wiping, smudging, painting, smearing, blotting, soaking, and spraying.

Dendrimer and/or Hyperbranched Compounds

Dendrimers and hyperbranched compounds are well known in the prior art. Hyperbranched compounds are molecular constructions having a branched structure, generally around a core. Their structure generally lacks symmetry. The branches can be of different natures and lengths. Dendrimers are highly branched having a well-defined chemical structure. As a general rule, dendrimers comprise a core, a given number of generations of branches, or spindles, and end groups. The generations of spindles consist of structural units which are identical for the same generation of spindles and which may be identical or different for different generations of spindles. The generations of spindles extend radially in a geometrical progression from the core.

Dendrimers or hyperbranched compounds useful in the invention are typically polymers or oligomers. In hyperbranched polymers, the number of base units, or monomers, may be different depending on the different branching. While at the same time being asymmetrical, hyperbranched polymers can have: an extremely branched structure, around a core; successive generations or layers of branching; a layer of end chains.

Hyperbranched polymers are generally derived from the polycondensation of one or more monomers ABx, A and B being reactive groups capable of reacting together, x being an integer greater than or equal to 2, but other preparation processes may be envisaged. Hyperbranched polymers are characterized by their degree of polymerization DP=1−b, b being the percentage of non-terminal functionalities in B which have not reacted with a group A. Since the condensation is not systematic, in contrast with the synthesis of dendrimers, the degree of polymerization is less than 100%. An end group T can be reacted with the hyper-branched polymer to obtain a particular functionality on the ends of chains.

Several hyperbranched polymers can be combined together, via a covalent bond or another type of bonding, by means of their end groups. Such polymers, known as bridged polymers, fall within the definition of the hyperbranched polymers according to the present invention.

The definition of dendrimers given above includes molecules containing symmetrical branching; it also includes molecules containing non-symmetrical branching, such as, for example, dendrimers whose spindles are lysine groups, in which the branching of one generation of spindles on the preceding generation takes place on the α and ε amines of lysine, which leads to a difference in the lengths of the spindles of different branching.

Dense star polymers, starburst polymers and rod-shaped dendrimers are included in the present definition of dendrimers. The molecules known as arborols and cascade molecules also fall within the definition of dendrimers according to the present invention.

Several dendrimers can be combined together, via a covalent bond or another type of bonding, by means of their end groups to give species known as bridged dendrimers or dendrimer aggregates. Such species are included in the definition of dendrimers according to the present invention.

Dendrimers can be in the form of an assembly of molecules of the same generation, which are referred to as monodisperse assemblies; they can also be in the form of assemblies of different generations, known as polydisperse assemblies. The definition of dendrimers according to the present invention includes both monodisperse and polydisperse assemblies of dendrimers.

Dendritic polymers and hyperbranched polymers are prepared by condensation reactions of monomeric units having at least two different types of reactive groups and many are commercially available for use in cosmetic products. The preparation of dendrimers has been described in the art, see, e.g., U.S. Pat. Nos. 4,289,872 and 4,410,688, which describe dendrimers based on layers of lysine units).

A group of polymers useful in the present invention are hyperbranched polyesteramides having at least one quarternized amine end-group. Hyperbranched polyesteramides having quaternized amine end groups suitable for the purpose of the present invention are disclosed in WO2007098888, in particular on page 3, line 9 to page 11, line 10 which is incorporated herein by reference.

Preferably, the amine end groups of the hyperbranched polyesteramides are quaternized using linear or branched (C₁-C₈) alkyl groups, in particular linear saturated (C₁-C₈) alkyl groups such as in particular with methyl and/or ethyl groups or they are quaternized using benzyl groups. The counter ion normally results from the quaternization agent but can be exchanged if necessary. Preferably, the counter ion is chloride and/or MeSO₄⁻ and/or EtSO₄⁻ in particular, the counter ion is MeSO₄⁻ and/or EtSO₄⁻. Other quaternization agents are epoxides like cyclohexene oxide and glycidyl ethers and esters like glycidol and glycidylmethacrylate. Suitable agents to obtain a betaine group are chloro acetic acid and (meth)acrylic acid. In all embodiments of the invention, the preferred amine end groups are quaternized di(m)ethylaminopropyl groups or N-methylpiperazinyl groups which are quaternized with methyl or ethyl groups, most in particular the quaternized amine end groups are tri (m)ethylammoniumpropyl groups.

In certain embodiments, the quaternized amine end-groups can be selected from quaternized di(m)ethylaminoethyl, di(m)ethylaminopropyl, di(m)ethylaminohexyl and/or N-methylpiperazinyl end groups. In particular the quaternized amine end groups are tri (m)ethylammoniumpropyl groups.

The amine end-groups are preferably quaternized with dimethylsulfate and/or diethylsulfate. Most preferably, the quaternized amine end groups are tri (m)ethylammoniumpropyl groups

Exemplary amine end groups are di(m)ethylaminopropyl or N-methylpiperazinyl are quaternized with 3-chloro-2-hydroxypropyltrimethylammonium chloride.

Synthesis of particular exemplary hyperbranched polyesteramide polymers can be found in Examples 1 and 2 of US 2011/0165107 and Example 1 of US 2011/0182843, the contents of which are incorporated herein by reference.

Hyperbranched compounds can also include the use of highly branched alpha-D-glucans as described in U.S. Pat. No. 10,117,937, such as hyperbranched amylopectins and derivatives thereof. Amylopectins are very generally branched starches or starch products.

A degree of branching can be defined for amylopectins. The measure of branching is the ratio of the number of anhydroglucose molecules bearing branch points (α-(1-6) bonds) to the total number of molecules the anhydroglucose of amylopectin, this ratio being expressed in mol %. Amylopectin occurring in nature has degrees of branching of approximately 4 mol %. However, it is known that clusters and molecular sections of amylopectin have a slightly higher degree of branching when viewed in isolation than the average degree of branching by nature. Hyperbranched amylopectins in the sense of the invention are those amylopectins which have a degree of branching which goes significantly beyond the degree of branching known from nature for amylopectins. The degree of branching is in any case an average (mean degree of branching) since amylopectins are polydisperse substances.

Such hyperbranched amylopectins have significantly higher degrees of branching, expressed as mol % of the branching anhydroglucoses, compared to unchanged amylopectin or hydroxyethyl starch and are therefore more similar in structure to glycogen.

As another example, the dendrimer or hyperbranched compound may include one or more poly alpha-olefin oils. Non-limiting examples of poly α-olefin oils can include polydecenes having a number average molecular weight of about 500, having a number average molecular weight of about 3000, and having a number average molecular weight of about 6000.

Dendrimers can further include branched organopolysiloxanes, an example of which is Lauryl PEG-10 Tris(Trimethylsiloxy) silylethyl Dimethicone.

As another example, the dendrimer can be a polylysine dendrimer as described in U.S. patent application Ser. No. 62/823,730, U.S. Pat. No. 4,289,872 and US Patent Application Publication No. 2008/0206183, the contents all of which are incorporated herein by reference. In some embodiments, the polylysine dendrimer is an unconjugated poly-L-lysine dendrigraft of second generation having a DPn from 40 to 60, preferably from 46 to 50, such as 48.

In some other embodiments, the polylysine dendrimer is a poly-L-lysine dendrigraft having a linear poly-L-lysine core wherein the e-amino groups are substituted with a poly-L-lysine moiety. For instance, the linear poly-L-lysine core contains from 6 to 10, preferably 8 lysine residues in length and/or the poly-L-lysine moiety grafted on e-amino groups have from 2 to 6, preferably 5 lysine residues in length.

In some further embodiments, the polylysine dendrimer is in the form of a polycationic dendrimer wherein the counter anion is acetate.

Preferably, the dendrimer or hyperbranched compound is selected from the group consisting of polylysines, amylopectins, poly alpha-olefins, organopolysiloxane copolymers, polyquaterniums, and combinations thereof.

In certain particularly preferred embodiments, the dendrimer or hyperbranched compound is poly-L-lysine and/or lauryl PEG-10 Tris(Trimethylsiloxy) silylethyl Dimethicone and/or amylopectin.

In other particularly preferred embodiments, the dendrimer or hyperbranched compound is a polyolefin, such as Hydrogenated Poly(C6-14 Olefin).

Most preferably, the polylysine is a poly-L-lysine dendrimer, such as a poly-L-lysine dendrigraft. Dendrigraft polymers have a less controlled structure than conventional dendrimers (e.g., as described in U.S. Pat. No. 4,289,872), but usually a more regular architecture compared to hyperbranched polymers (e.g., as described in Rodriguez-Hemandez, et al. (2003) Biomacromolecules 4:249-258 and Klok, et al. (2002) Macromolecules 35:8718-8723). poly-L-lysine dendrigraft for use in this invention can be prepared as described Example 1 of WO2020197669, or as described, e.g., in US 2008/0206183 or Collet, et al. (2010) Chem. Eur. J. 16:2309-16.

In certain preferred embodiments, the poly-L-lysine dendrimer is provided in a mixture with glycerin and water.

### Primer Composition

The primer composition will comprise one or more of the above-described dendrimer and/or hyperbranched compounds.

Primer compositions of the invention are typically “rinse-off” compositions to be applied to the hair and then rinsed away, such as a shampoo.

The dendrimer/hyperbranched compound-containing primer composition preferably is shampoo comprising one or more surfactants. As used herein, the term “surfactant,” includes compounds that may lower the surface tension, or interfacial tension, between two liquids or between a liquid and a solid. Surfactants may act as detergents, wetting agents, emulsifiers, foaming agents, and dispersants.

Other additives may be included in the primer composition, such as those ordinarily found in personal care and/or textile care formulations. Such additives may also include sensates, fragrances, colorants, metal chelators, pigments, water, oil, waxes, alcohols, surfactants, UV filters, light reflectants or dispersants, glitter, conditioners, and preservatives.

The level of dendrimer or hyperbranched compound in the primer composition is from 0.005 to 5 wt % of the total composition, preferably the level is from 0.0.01 to 2.75 wt %, more preferably from 0.02 to 2.5 wt %. In certain embodiments, the dendrimer is about 0.04% to about 1.375% by weight of the primer composition.

A preferred form of adding the dendritic macromolecule to the composition is to add the macromolecule together with any surfactant and/or long chain alcohol.

### Shampoo

In addition to dendrimer or hyperbranched compound, the primer composition can be a shampoo comprising one or more nonionic surfactants, which can be included in an amount ranging from 0.5 to 8 wt.-%, preferably from 1 to 5 wt. % active nonionic surfactant based on the total weight of the preparation.

For example, representative nonionic surfactants that can be included into primer compositions (shampoos) according to the invention include alkyl polyglycosides (APGs). Typically, the APG is one which comprises an alkyl group connected (optionally via a bridging group) to a block of one or more glycosyl groups such as e.g., Decyl Glucoside; Coco-Glucoside, Lauryl Glucoside, and Caprylyl/Capryl Glucoside.

Other representative nonionic surfactants include mono- or di-alkyl alkanolamides such as e.g., coco mono- or di-ethanolamide and coco mono-isopropanolamide.

Examples of suitable anionic surfactants that may be included in the primer compositions are alkyl sulfates, alkyl ether sulfates, alkylaryl sulphonates, alkanoyl isethionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulfates, alkyl ether sulphosuccinates, alkyl ether phosphates and alkyl ether carboxylic acids and salts thereof may contain from 1 to 20 ethylene oxide or propylene oxide units per molecule.

In particular, the anionic surfactants are preferably selected from sodium lauryl methyl isethionate, sodium methyl 2-sulfolaurate, sodium methyl cocoyl taurate, disodium 2-sulfolaurate and mixtures thereof.

In particularly preferred embodiments, the primer composition (shampoo) is free of sulfates, including the above referenced alkyl sulfates and alkyl ether sulfates, and in particular do not incorporate sodium lauryl sulfate (SLS) or sodium laureth ether sulfate (SLES).

The total amount of the anionic surfactant in the primer composition according to the invention ranges from 0.5 to 25 wt.-%, preferably from 1.5 to 15 wt.-%, more preferably from 2 to 14 wt.-%, in particular from 4 to 8 wt.-% of active anionic surfactant based on the total weight of the composition.

The primer composition preferably includes co-surfactants, to help impart aesthetic, physical or cleansing properties to the compositions.

Examples of co-surfactants are amphoteric or zwitterionic surfactant, which can be included in an amount ranging from 0.5 to about 8 wt.-%, preferably from 2 to 6 wt.-% active based on the total weight of the shampoo preparation. Examples of amphoteric or zwitterionic surfactants include alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoo preparations according to the invention include lauryl amine oxide, cocodimethyl sulphopropyl betaine, lauryl betaine, cocamidopropyl betaine (CAPB), sodium cocoamphoacetate and disodium cocoamphodiacetate. A particularly preferred amphoteric or zwitterionic surfactant to be used in the shampoo preparations of the present invention is cocamidopropyl betaine.

Mixtures of any of the foregoing amphoteric or zwitterionic surfactants may also be suitable. Preferred mixtures are those of cocamidopropyl betaine with further amphoteric or zwitterionic surfactants as described above such as in particular with sodium cocoamphoacetate.

It has been found that shampoo preparations comprising an additional amount of an amphoteric or zwitterionic surfactant are in particular suitable in the system for increasing volume. Thus, in a particular preferred aspect the invention relates to a shampoo preparation comprising nonionic surfactant, anionic surfactant, and an amphoteric or zwitterionic surfactant.

The total amount of surfactant (including any co-surfactant, and/or any emulsifier) in a shampoo preparation according to invention is generally from 1 to 50%, preferably from 5 to 30%, more preferably from 10 to 20%, in particular from 12 to 15 wt.-% based on the total weight of the composition.

The primer compositions according to the invention may also contain cationic polymers for further enhancing conditioning performance. Suitable cationic polymers may be homopolymers which are cationically substituted or may be formed from two or more types of monomers. The weight average molecular weight (Mw) of the polymers will generally be between 100 000 and 2 million Daltons. Suitable cationic polymers include, for example, copolymers of vinyl monomers having cationic amine or quaternary ammonium functionalities with water soluble spacer monomers such as (meth) acrylamide, alkyl and dialkyl (meth) acrylamides, alkyl (meth) acrylate, vinyl caprolactone and vinyl pyrrolidine.

An additional component of compositions as contemplated herein is a cationic guar polymer. Suitable cationic guar polymers are available from natural sources and give the hair a soft feel. They furthermore support the separation of the oils on the hair without negatively influencing the hair volume in the care active substance mixture of the hair cleaning agents as contemplated herein.

In the context of the present disclosure, suitable cationic guar polymers are understood to mean physiologically compatible cationic guar derivatives and/or hydrophobically modified cationic guar derivatives.

Preference is given to cationic hydroxy (C₁-C₄) alkyl guar derivatives, preferably cationic hydroxyethyltrimethylammonium guar and/or cationic hydroxypropyltrimethylammonium guar having average molecular weights (weight average) from about 100,000 to about 2,000,000 daltons, preferably from about 200,000 to about 1,750,000 daltons and in particular from about 300,000 to about 1,600,000 daltons. Further preferred are cationic hydroxy (C₁-C₄) alkyl guar derivatives, preferably cationic hydroxyethyltrimethylammonium guar and/or cationic hydroxypropyltrimethylammonium guar, having cationic charge densities of at least about 0.5 meq/g.

Especially preferred are the cationic guar polymers known under the INCI name Guar Hydroxypropyltrimonium Chloride having a molecular weight (weight average) from about 100,000 to about 2,000,000 daltons, preferably from about 200,000 to about 1,750,000 daltons and in particular from about 300,000 to about 1,600,000 daltons and a cationic charge density of at least about 0.5 meq/g.

In order to further increase the nourishment and/or rheological properties of the agents as contemplated herein it can be advantageous if they also contain at least one cosmetic oil. Suitable cosmetic oils in the sense of the present disclosure are understood to mean oil bodies that have a melting point below about 50° C., particularly preferably below about 47° C., very particularly preferably below about 44° C., most preferably below about 40° C. Most preferred are cosmetic oils which are flowable at a temperature below about 40° C.

Preferred cosmetic oils are natural vegetable oils. Vegetable oils (and/or butters)—even when used at low concentrations—can further increase the aforementioned conditioning advantages on the hair in combination with the active substance combination a), b), c), without a buildup effect occurring with regular use. In addition, vegetable oils (and/or butters) in the nourishing active substance mixture of the hair treatment agents as contemplated herein also contribute to the improvement in hair shine.

Examples of natural vegetable oil suitable as contemplated herein are, for example, amaranth seed oil, apricot kernel oil, argan oil, avocado oil, babassu oil, cottonseed oil, borage seed oil, camelina oil, canola oil, cranberry oil, safflower oil, peanut oil, pomegranate seed oil, grapefruit seed oil, hempseed oil, rose hip oil, hazelnut oil, elderberry seed oil, blackcurrant seed oil, jojoba oil, cocoa butter, coconut oil, pumpkin seed oil, linseed oil, macadamia nut oil, corn oil, mallow oil, almond oil, mango stone oil, marula oil, poppy seed oil, evening primrose oil, olive oil, palm oil, palm seed oil, peach stone oil, rambutan oil, rapeseed oil, rice bran oil, castor oil, Sacha Inchi oil, safflower oil, seabuckthorn berry oil, seabuckthorn kernel oil, sasanqua oil, sesame oil, Shea butter, soybean oil, sunflower oil, teatree oil, grape seed oil, tsubaki oil, walnut oil, wheatgerm oil, lady's smock oil and/or wild rose oil.

Preferred are amaranth seed oil, apricot kernel oil, argan oil, avocado oil, coconut oil, almond oil, macadamia nut oil, rose hip oil, sunflower oil, olive oil, peach kernel oil, jojoba oil and/or the vegetable butters Shea butter and/or cocoa butter. In certain preferred embodiments, a hydrolized jojoba ester is utilized.

In a further preferred embodiment, the primer composition contemplated herein is substantially free from silicones, and/or mineral oils. The term “substantially free” is understood to mean that the compositions as contemplated herein preferably contain less than about 0.25% by weight of, more preferably less than about 0.10% by weight of, and in particular no silicones and/or mineral oils (in relation to the total weight of the hair cleansing agents). The aforementioned amounts apply here both for freely added silicone and/or mineral oil and for silicones and/or mineral oils which might be contained in trade products as by-product.

In a further preferred embodiment, the primer composition contemplated herein is substantially free from polymeric thickeners of synthetic and/or natural origin. The term “substantially free” is understood to mean that the hair treatment agents as contemplated herein preferably contain less than about 0.25% by weight of, more preferably less than about 0.10% by weight of, and in particular no polymeric thickeners of synthetic and/or natural origin (in relation to the total weight of the hair cleansing agents). The aforementioned amounts apply here both for freely added polymeric thickeners of synthetic and/or natural origin and for polymeric thickeners of synthetic and/or natural origin which might be contained in trade products as by-product.

Further active substances, auxiliaries and additives which can preferably be present in primer compositions (shampoos) as contemplated herein are, for example: plant extracts, humectants, perfumes, UV filters, structurants such as maleic acid, dimethyl isosorbide, cyclodextrins, fiber-structure-improving active substances, in particular mono-, di- and oligosaccharides such as, for example, glucose, galactose, fructose, fruit sugar and lactose, dyes for staining the agent, active substances such as bisabolol and/or allantoin, complexing agents such as EDTA, NTA, beta-alanine diacetic acid and phosphonic acids, ceramides (Ceramides are understood to mean N-acylsphingosine (fatty acid amides of sphingosine) or synthetic analogues of such lipids (so-called pseudo-ceramides)), propellants such as propane-butane mixtures, N₂O, dimethyl ether, CO₂ and air, antioxidants, preservatives, such as sodium benzoate or salicylic acid, additional viscosity regulators, such as salts (NaCl).

In embodiments of the invention, it may be preferred that the pH of the primer composition is about 3.0 to 6.0 or about 4.3 to about 5.3. To arrive at this pH, various alkali compounds may be added to adjust the composition as is known in the art. Such alkali compounds may include ammonia, sodium hydroxide, potassium hydroxide, monoethanolamine, diethanolamine, diisoproanolamine, triethanolamine, and isopropanolamine.

The primer composition may be prepared in any dispensed product format desired or known in the art. For example, it may take the form of a liquid, semi-liquid, gel, spray, mist, foam, mousse, paste, crème, emulsion, and the like. In some embodiments, the composition is prepared as a loose or compressed powder, granules, pellets, etc., to which the end consumer or the distributor may add a liquid or semi-liquid solvent or carrier. Such forms may be desirable to reduce shipping costs (less weight to transport).

Primer compositions can have a viscosity ranging from 120,000 cps to 3,000,000 cps. Viscosity can be measured using suitable viscometer known to those of skill in the art. The preferred viscosity range is 5,000 cps-15,000 cps using a Brookfield LV Viscometer, Spindle 3 at 6 rpm for 60 sec at 25° C., with around 10,000 cps being the most preferred.

The primer composition can be filled and stored in a wide variety of containers. Suitable containers included, but are not limited to, glass or plastic beakers and bottles, tubes, aluminum or plastic jars and tubes, blister packs and other monodose type containers. Suitable plastics include PP, PS, PET, LDPE, HDPE, and combinations thereof. The compositions are not limited by the type of container but are advantageous in that they are able to utilize a wider variety of packaging than traditional and bar shampoo products

The primer compositions of the invention are used as follows. The product is applied to hair that has been wetted or which will be subsequently wetted. The user will distribute the product throughout the hair mechanically in the usual way of spreading creams and lotions, by rubbing the product with his/her hands or combing through the hair. The compositions, when applied to wetted hair, will foam. The user will massage the hair and scalp, and then the hair is rinsed with water to remove dirt, oils, and the majority of the compositions from the hair.

Since the amount of hair on each consumer varies greatly and is user dependent, the amount of primer composition to use is also consumer dependent. However, it is noted that there is no “mixing” needed prior to application to hair; the product is directly applied to wet hair.

### Chitosan Dehydroxanthan Gum Complex Compositions

The compositions and methods further utilize a chitosan complex composition, which can form a film over hair or other fibers previously treated with the primer composition containing a dendrimer or a hyperbranched compound. The chitosan complex composition preferably comprises chitosan, a dendrimer and chitosan complexing agent (e.g., dehydroxanthan gum), an aldehyde-bearing compound (ABC), and an organic acid.

Any chitosan may be used. Preferably, the chitosan is high density and low molecular weight, such as those available under CAS 9012-76-4.

In an embodiment, it may be preferred that chitosan having a deacetylation of about 50% to about 100%, about 60% to about 80% and about 70% to about 95% is used, and molecular weights of about 50,000 to 1,000,000 g/mole, 300,000 to 2,000,000 g/mole and 500,000 to 5,000,000 g/mole.

The complex composition with typically comprise to 0.1 to about 10% by weight chitosan, preferably 0.3 to 3% by weight, more preferably 0.4 to 1.5% by weight, most preferably 0.5 to 1.0% by weight.

The complex composition further includes a dendrimer or hyperbranched compound and chitosan complexing agent, preferably which is dehydroxanthan gum.

In certain embodiments, Sodium Polyitaconate may be used instead of, or in conjunction with, the dehydroxanthan gum.

The complex composition described herein comprises at least 0.1%, alternatively at least 0.6%, alternatively at least 0.65%, alternatively at least 0.75%, alternatively at least 1.0%, alternatively from about 0.1% to about 3%, alternatively from about 0.2% to about 2.0%, alternatively from about 0.5% to about 1.5%, alternatively from about 0.8% to about 1.2% complexing agent, by weight of the composition.

The complex composition further includes an aldehyde-bearing compound (ABC). By ABC it is meant any compound having one or more aldehyde functional groups that are available to react in situ on the fiber with the nitrogen atoms of chitosan. In some embodiments, it may be preferred that the ABC is vanillin, p-vanillin, o-vanillin, or ethylvanillin. Other suitable ABCs may include, for example without limitation, benzaldehyde, formaldehyde, acetaldehyde, (R)-carvone, cinnamaldehyde, glyoxylic acid, 3-methyl-3 phenyl glycidic acid ethyl ester, acetaldehyde, capraldehyde, piperonal citral, undecanal, octanal, heptanal, nonanol, dodecanal, ethyl 3-(4-methylphenyl) oxirane-2-carboxylate, unadecalactone, oleic aldehyde, glyoxal, glutaraldehyde, and methyl nonyl acetic aldehyde. Combinations of different ABCs may also be used in the composition.

The chitosan complex composition will typically comprise to 0.1 to 2.0% by weight ABC, more preferably 0.5 to 1.0% by weight, most preferably 0.75 to about 0.85% by weight.

The complex composition also includes an organic acid such as a mono- or dibasic acid. Any may be suitable. In some embodiments, the preferred organic acid is acetic acid, most preferably glacial acetic acid. Other organic acids that may be used include, without limitation, formic acid, glycolic acid, lactic acid, ascorbic acid, pyruvic acid, salicylic acid, citric acid, adipic acid, succinic acid, malic acid, tartaric acid, fumaric acid, glyoxilic acid and combinations thereof.

In certain embodiments, the chitosan complex composition may additionally include cosmetically acceptable carriers and adjunct ingredients.

As an example, the chitosan complex composition may further include at least 20% of one or more aqueous carriers, by weight of the hair care composition. According to an embodiment, the aqueous carrier may be prepared from demineralized or distilled water. Other acceptable carriers that may be used in the aqueous carrier include, but are not limited to, alcohol compounds, such as ethanol. According to an embodiment, the composition comprises alcohol, dipropylene glycol, and/or water.

The chitosan complex compositions may comprise a rheology modifier to increase the substantivity of the composition. Any suitable rheology modifier can be used. In an embodiment, the chitosan complex composition may comprise from about 0.1% to about 10% of a rheology modifier, alternatively from about 0.5% to about 2.2% of a rheology modifier, alternatively from about 0.7% to about 2% of a rheology modifier, alternatively from about 1% to about 1.7% of a rheology, and alternatively from about 1% to about 1.5% of a rheology modifier, by weight of the composition. In an embodiment, the rheology modifier may be a polyacrylamide thickener.

In some embodiments of the invention, the complex composition may include a component that provides a specialized attribute to the final film, for example, pigments or colorants to impart color to the fiber or hair, UV filters for sunscreen or to increase color protection, light reflectants or dispersants to enhance shine.

Other additives may be included in the composition, such as those ordinarily found in personal care and/or textile care formulations. Such additives may also include sensates, fragrances, colorants, metal chelators, pigments, water, oil, waxes, alcohols, surfactants, UV filters, light reflectants or dispersants, glitter, conditioners, and preservatives.

Preferably, the chitosan complex composition is free of silicone or silicone polymers.

In embodiments of the invention, it may be preferred that the final pH of the composition is about 2.0 to 5.0 or about 3.5 to about 4.5. To arrive at this pH, various alkali compounds may be added to adjust the composition as is known in the art. Such alkali compounds may include ammonia, sodium hydroxide, potassium hydroxide, monoethanolamine, diethanolamine, diisoproanolamine, triethanolamine, and isopropanolamine.

The composition may be prepared in any dispensed product format desired or known in the art. For example, it may take the form of a liquid, semi-liquid, gel, spray, mist, foam, mousse, paste, crème, emulsion, and the like. In some embodiments, the composition is prepared as a loose or compressed powder, granules, pellets, etc., to which the end consumer or the distributor may add a liquid or semi-liquid solvent or carrier. Such forms may be desirable to reduce shipping costs (less weight to transport).

In the practice of an embodiment of the invention, a chitosan composition containing any of the components described above is prepared and applied topically to hair or fiber(s). Optionally, the hair or fiber(s) is then rinsed with water. The hair or fiber is then allowed to dry. Preferably, heat is applied to the fiber—preferably at a temperature of about 50° C. or greater, such as with a hair dryer. Alternatively, the temperature is about 60° C. to about 190° C. or about 100° C. to about 160° C.

The heat may be applied by any means known or developed in the art. It may be applied directly or ambiently. Suitable devices for providing heat include, without limitation, a hair dryer, a blow dryer, blower, a diffuser, and combinations thereof.

In general, it may be preferred that the fiber is exposed to the heat for duration of time (“drying time”) sufficient to drive off at least portion of the aqueous solvent from the fiber. In some embodiment, this may mean that the fiber is about 60% to about 100% dry, about 70% to about 95% dry, and/or about 80% to about 90% dry. The drying times to achieve these percent drynesses will vary. As an example, in the case of a head of shoulder length hair of average thickness, the drying time may be about 2 minutes, 5 minutes, about 10 minutes, or about 20 minutes to achieve these results.

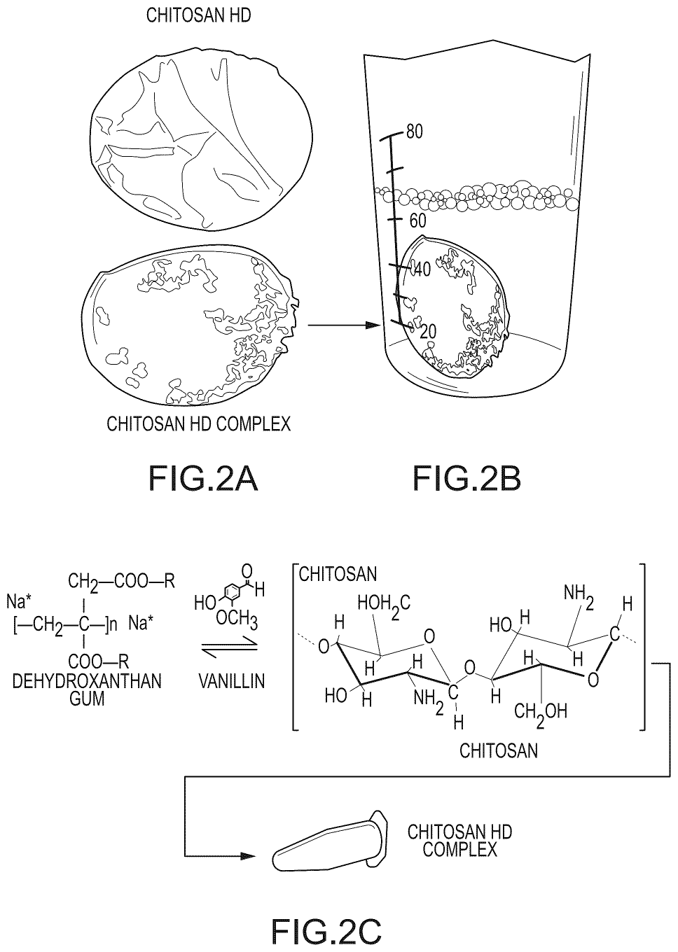

Upon removal of at least a portion of the aqueous solvent, a chitosan film will be stably coated on the fiber. Without binding themselves to a theory, the inventors propose that the film is formed by in the manner suggested in FIG. 2C.

As a consequence of the adhesion of the chitosan complex of the invention to fiber, especially mammalian hair, the films formed and the effects provided by the films are, in an embodiment, semi-permanent. By semi-permanent it is meant that the film is present on the hair or fiber and/or the effect provided by the film to the fiber or hair (“film effects”) is observable after the hair is washed in a conventional manner at least 3 times or more. For example, the film or the film effects may be observable for about 10 to about 40 washings. The degree of permanency will vary depending on the specific hair type and the end effect for which the film is applied.

In the practice of an embodiment of the invention, a chitosan complex composition containing any of the components described above is prepared and applied topically to the surface of the fiber or is added to a cosmetic carrier composition which is then applied topically to hair after a dendrimer primer composition described herein has been applied to the fiber.

The methods may be used to provide color protection (that is, reduce or prevent leaching of color from hair that has been previously dyed, thereby increasing the time between hair coloring or recoloring sessions). The reduction in color leaching or loss is observable and is quantifiable using the color metric set forth by the International Commission on Illumination. Under such metric, hair or fiber coated with the film of the invention may have, for example, a ΔE of about 6.5 or less after 10 washings, more preferably less than 4.0, most preferably less than 2.0 after ten washings, or a ΔE of about 8.0 or less after 20 washings, more preferably less than 4, most preferably less than 2.5 after 20 washings, or a ΔE of about 9.0 or less after 30 washings, more preferably less than 6, most preferably less than 4.5 after 30 washings, or a ΔE of about 10.0 or less after 40 washings, more preferably less than 5.5, most preferably less than 5 after 40 washings.

In an embodiment, a benefit provided by the combination of primer composition and chitosan complex described herein on a fiber includes increasing or enhancing the fiber's ability to retain color dye over time, especially acid dyes, such as Acid Black, Acid Blue, Acid Green, Acid Red, Acid Violet, Acid Yellow, and Acid Orange dyes. The acid dyes are water soluble or in ethanol and are highly anionic due to their sulfonic acid groups. Other dyes that may be used include acid leveling dyes. The color dye may be for a textile fiber or for a hair.

As an example, relating to hair coloring, semi-permanent or permanent hair coloring is performed by first applying to the hair an aqueous or aqueous/alcoholic solution of the dye(s) at pH less <8 (potentially more preferred at pH 4-6). The dye solution remains on the hair for about 20 minutes, depending on the condition of hair. The hair is then rinsed with water. The hair may then be left wet, and then treated with the primer composition followed by the chitosan complex composition as described above, or alternatively, dried, then rewetted and treated with the primer composition followed by the chitosan complex composition as described above. The hair is then dried, optionally with heated air as described above.

It is believed that the ionic interactions of the sulfonic groups —(SO₃) of the acid dyes and the —NH₃⁺ of the chitosan complex composition are strong, resulting in long lasting uniform semi-permanent hair color lasting over several shampoos compared to colored hair without the polymer film.

The invention further extends to hair care assemblies and kits to treat, or otherwise deliver or deposit the foregoing compositions to hair. Such kits may include communication devices that provide information related to the kit, such as instructions. These devices may take any form, such as a writing, video, audio, etc.

The kits and assemblies may also include accessory useful in the use or application of the contents of the kit or hair care generally. Such accessories can include gloves, a hair clip, a comb, a plastic cap or hat, a timer, a swab, a brush, and a mirror.

### EXAMPLES

### Example 1: Chitosan Complex

Several chitosan mixtures were prepared containing water, Chitosan HD, vanillin, and glacial acetic acid. Dehydroxanthan gum was added to one of the mixtures and Sodium Polystyrene Sulfonate to another. The chitosan was weighed and added slowly into the glacial acetic acid. The mixture was stirred until the chitosan dissolved. The vanillin was added and mixed further until all vanillin dissolved. Dehydroxanthan gum or Sodium Polystyrene Sulfonate was thereafter added.

The composition using dehydroxanthan gum was a cloudy, homogeneous solution whereas use of Sodium Polystyrene Sulfonate as a complexing agent formed a precipitate and there was visible phase separation of the composition.

Films of the base composition and the base with dehydroxanthan gum were cast in a petri dish by pouring the mixture in the petri dish and letting the film dry overnight. Infrared spectra were obtained.

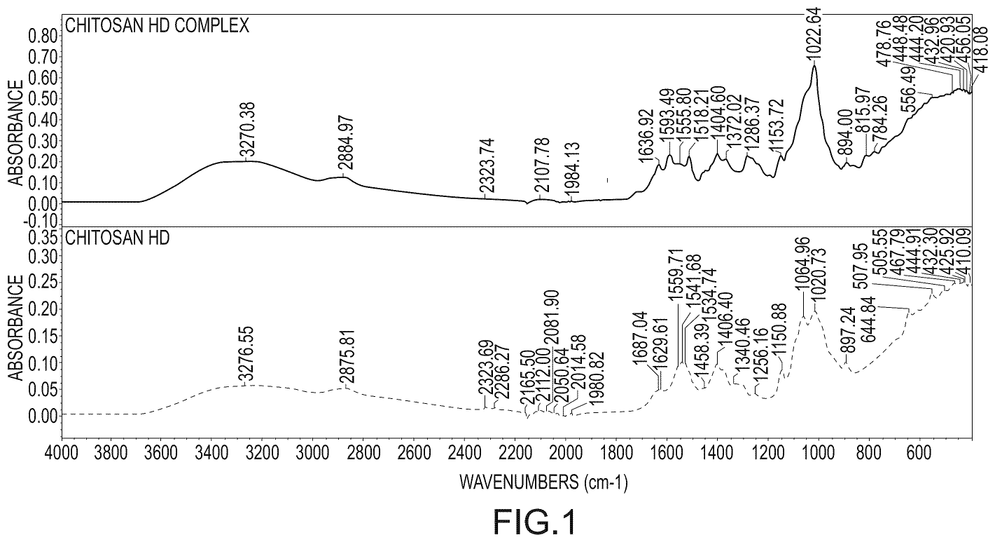

FIG. 1 shows the FT-IR spectrum of the base chitosan composition in the lower plot, and the FT-IR spectrum of the base chitosan composition with dehydroxanthan gum shown in the upper plot. There is a visible shift from 1559.74 cm 1 to 1593.66 cm 1 stretch in the FT-IR spectrum of the base chitosan composition with dehydroxanthan gum, which is believed to signify N—H bending. Without wishing to be bound by theory, this shift in N—H bending could be a result of the N—H groups from the chitosan complexing with dehydroxanthan gum with the aid of a vanillin crosslinker. A similar shift was observed by Cai, L., Shi, H., Cao, A. et al. Characterization of gelatin/chitosan polymer films integrated with docosahexaenoic acids fabricated by different methods. Sci Rep 9, 8375 (2019).

The film comprising dehydroxanthan gum was placed in a 1% solution of shampoo in water and did not dissolve until after approximately 30 minutes of stirring on a stir plate. FIGS. 2A and 2B show images of the film before and after being placed in the shampoo solution.

### Example 2: Dendrimer Shampoo Compositions

Five hyperbranched/dendrimer compounds were added to a base shampoo composition (Base 2B) to create five “dendrimer shampoo” compositions. The shampoo base composition ingredients are listed below in order of decreasing amount. The base contained 13% total active surfactant comprising a mixture of non-ionic, amphoteric, and anionic surfactant compositions. The non-ionic surfactant was added first, then amphoteric and finally the anionic surfactants to prevent any precipitates from forming. The dendrimers utilized are described in TABLE 1 and were added to the base composition at a concentration of 2.5% by weight.

The components of Base 2B were: Water, cocamidopropyl betaine, lauryl glucoside, glycerin, sodium lauroyl methyl isethionate, sodium methyl 2-sulfolaurate, sodium methyl cocoyl taurate, caprylyl/capryl glucoside, disodium 2-sulfolaurate, fragrance, polyquaternium-10, levulinic acid, hydrolyzed jojoba esters, sodium gluconate, benzoic acid, benzyl alcohol, citric acid, sodium benzoate, sorbic acid, glucose, decyl alcohol, caprylyl alcohol, polylysine.

TABLE 1
Dendrimer/Hyperbranched Compounds
Used in Shampoo Compositions

| | | | Solubility | |
|---|---|---|---|---|
| Cmpd. | INCI | Type | (water/oil) | Form |
| D1 | Polyquaternium-110 | Hyperbranched | water | Liquid |
| | | Cationic | | |
| D2 | Amylopectin | Hyperbranched | water | Powder |
| | | Non-Ionic | | |
| D3 | Hydrogenated | PAO | water | Liquid |
| | Poly(C6-14 Olefin) | (polyalpholefin) | | |
| D4 | Lauryl PEG-10 | Dendrimer | water | Liquid |
| | Tris(Trimethyl- | | | |
| | siloxy)silylethyl | | | |
| | Dimethicone | | | |
| D5 | Glycerin (and) Water | Dendrimer | water | Liquid |
| | (and) Polylysine | | | |

### Example 3: Shampoo Stability

The bubble count, foam volume and foam height of the dendrimer shampoos of Example 2 were measured using a KRUSS Scientific Dynamic Foam Analyzer DFA 100. Results are provided in TABLE 2. For comparison, the control (2A) was a commercially purchased Moisturizing Shampoo containing Water/Aqua/Eau, Sodium C14-16 Olefin Sulfonate, Decyl Glucoside, Cocamidopropyl Betaine, Dimethyl Lauramide/Myristamide, Glycerin, Phenoxyethanol, Elaeis Guineensis (Palm) Extract, Phalaenopsis Amabilis Extract, Cocos Nucifera (Coconut) Fruit Extract, Polyquaternium-10, Polyquaternium-47, Polyquaternium-7, Cetrimonium Chloride, Citric Acid, Glycol Distearate, Laureth-4, Sodium Gluconate, Sodium Hydroxide, Benzoic Acid, Sodium Benzoate, Fragrance/Parfum.

TABLE 2

| | | | Maximum | Time of |
|---|---|---|---|---|
| | Initial bubble | Final bubble | foam | maximum foam |
| | count per | count per | volume | height |
| Shampoo | (mm⁻²) | (mm⁻²) | (mL) | (s) |
| 2A | 143.77 | 129.04 | 130.8 | 16.38 |
| (Control) | | | | |
| 2B | 148.60 | 131.46 | 133.1 | 16.05 |
| 2B + D1 | 145.73 | 129.59 | 134.9 | 16.11 |
| 2B + D2 | 148.28 | 134.44 | 134.4 | 16.44 |
| 2B + D3 | 140.34 | 130.41 | 135.8 | 17.07 |
| 2B + D4 | 142.98 | 131.56 | 137.0 | 17.15 |
| 2B + D5 | 147.29 | 127.74 | 133.7 | 15.58 |

Table 2 shows that the initial and final bubble count do not change much indicating a stable foam in the presence of a dendrimer/hyperbranched compound in the shampoo. The maximum volume and the time it took to reach maximum volume was similar among all the shampoos with and without the dendrimer/hyperbranched compound.

### Example 4: Chitosan Serum

A chitosan complex emulsion (4S) was prepared by mixing together the ingredients in Table 3. The final pH was around 4.

TABLE 3
Composition 4S

| Ingredient | Wt. % |
|---|---|
| Chitosan HD | 0.8 |
| Dehydroxanthan Gum | 1.0 |
| vanillin | 0.2 |
| glacial acetic acid | 0.7 |
| rice vinegar | 1.0 |
| Propylene Glycol (and) Quaternium-26 | 1.0 |
| Preservative | 1.0 |
| Non-ionic solubilizer | 0.2 |
| Acrylates Copolymer | 6.0-8.0 |
| water | Qs |

### Example 5: Treatment and Styling with Shampoo+Serum

Various treatment combinations of dendrimer shampoo and chitosan serum were applied to mannequin heads to assess volumizing capability. The shampoo was applied first and rinsed off, then the chitosan serum was applied/sprayed on towel dry hair by using a comb to evenly spread and coat the hair with serum. The serum was left on the hair for 5 minutes before hair was blown dry with a round brush and CONAIR dryer set to high heat (about 80° C.) using 10 passes on each side over a 2 minute span. Thereafter, the hair was shampooed with a stripping shampoo and blown dry with the CONAIR dryer and a round brush a total of 10 times.

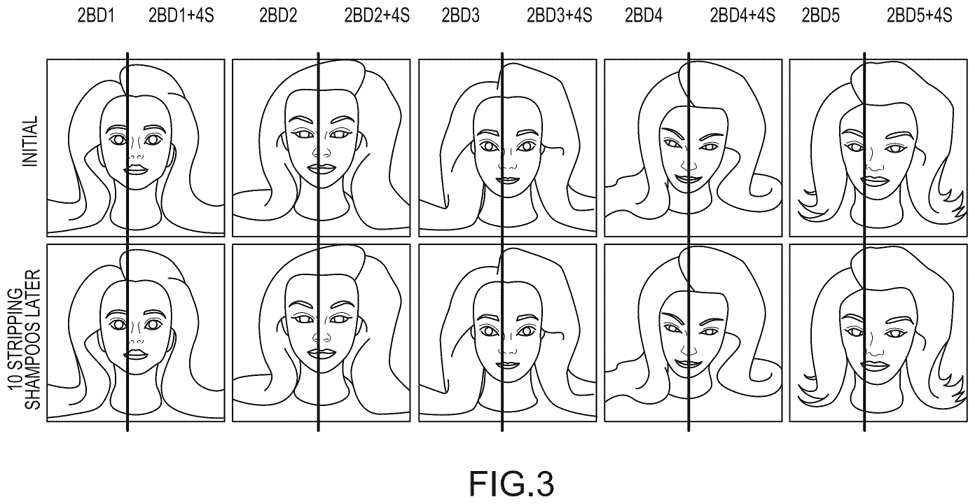

In FIG. 3, the left side of each mannequin contains the five dendrimer shampoos of Example 2 alone and the right side shows the combination of a dendrimer shampoo followed by application of the serum of Example 4 containing chitosan complex. The combination of dendrimer shampoo followed by chitosan complex serum shows a noticeable increase in volume for all five dendrimer/hyperbranched compounds utilized even after 10 stripping shampoos.

### Example 6: Retention of Treatment Compositions on Hair

For these experiments, single bleached swatches, as well as platinum bleached swatches were treated with a dendrimer shampoo primer composition and a chitosan complex composition, then washed 10 times with a stripping shampoo. The washed swatches were then stained with Direct Red 80 dye. Chitosan is known to adsorb Direct Red 80 dye, and the presence of dye retained on the swatches confirmed the presence of cationic chitosan HD complex on the hair fibers.

Specifically, the dendrimer shampoos of Example 2 were applied to the swatches. The chitosan serum emulsion of Example 4 was applied to the shampooed swatches at 0.4 g serum/1 g of hair. The serum was massaged into the hair and left on for 5 minutes, Then the hair was blown dry with a CONAIR blow dryer on high heat. The treated hair swatches were washed with commercially available Clarifying shampoo.

Direct Red 80 dye was applied to the swatches after the initial application on treatment(s) and after 10 washes with the stripping shampoo.

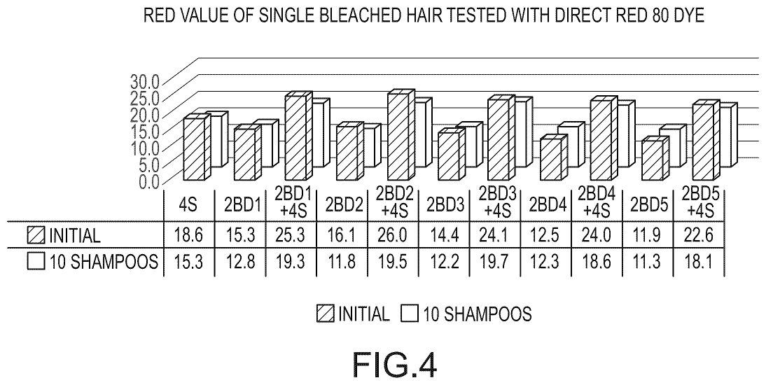

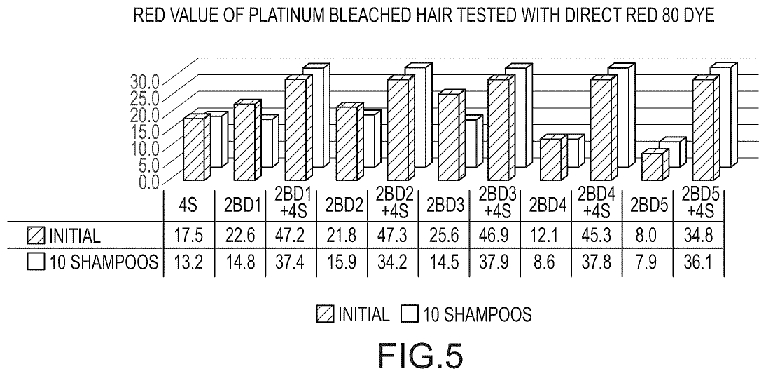

Hunter Color Coordinates (L A B values) were determined via laboratory measurement with a BYK color meter. An average of 3 measurements was used. Results are shown in FIG. 4 and FIG. 5. The bars represent the level of red on the swatches. As seen in FIGS. 4 and 5, the maximum red values were obtained in the samples which received both the primer composition and the chitosan complex composition. These samples had higher red values than samples which received treatment with only the primer composition or only the chitosan complex composition. Furthermore, the red values obtained after washing 10 times with a stripping shampoo were typically in the same range as the red values obtained during the initial state testing, although usually somewhat lower. The Red 80 dye tests confirm that (1) samples which received both the primer composition and the chitosan complex composition had a thicker, more volumizing coating of chitosan complex coating than the samples which received only the primer composition or the chitosan complex composition; and (2) the chitosan complex was not significantly degraded and remained on the hair for at least 10 washes.

### Example 7: Volume Analysis

For these experiments, brown virgin hair swatches were utilized in addition to the single bleached swatches and platinum bleached swatches of Example 6.

The swatches were treated with a dendrimer shampoo primer composition (Example 2) and a chitosan complex composition (Example 4).

Volume was analysed via IMAGEJ analysis initially and after 10 stripping shampoo washes using Clarifying shampoo with blow-dry.

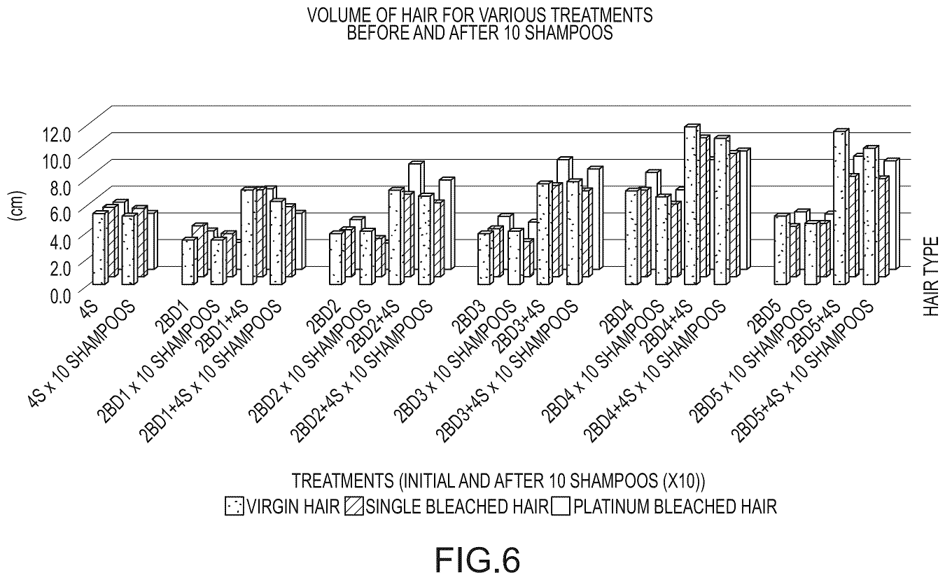

Results are provided in Table 4 and in FIG. 6, confirming that the combination of the primer composition and the chitosan complex composition is more effective at retaining volume over 10 washes with stripping shampoo as compared to the individual components alone. Volume was measured by using the largest width of the entire swatch of hair. An average of three measurements is provided.

TABLE 4
Volume Results

| | | Cm | | |
|---|---|---|---|---|
| | | Virgin | Single | Platinum |
| Formula | Time | Hair | Bleached | Bleached |
| Untreated hair | 0 | 3.1 | 2.96 | 2.8 |
| Serum (4S) | 0 | 5.5 | 5.4 | 5.2 |
| | 10 shampoos | 5.3 | 5.3 | 4.3 |
| shampoo 2BD1 | 0 | 3.5 | 3.9 | 3.0 |
| | 10 shampoos | 3.5 | 3.4 | 2.1 |
| shampoo 2BD1 + Serum | 0 | 7.2 | 6.7 | 6.2 |
| | 10 shampoos | 6.4 | 5.4 | 4.3 |
| shampoo 2BD2 | 0 | 3.6 | 3.6 | 3.8 |
| | 10 shampoos | 4.1 | 3.0 | 2.0 |
| shampoo 2BD2 + Serum | 0 | 7.3 | 6.4 | 8.0 |
| | 10 shampoos | 6.8 | 5.8 | 6.8 |
| shampoo 2BD3 | 0 | 4.0 | 3.7 | 4.1 |
| | 10 shampoos | 4.2 | 2.8 | 3.6 |
| shampoo 2BD3 + Serum | 0 | 7.7 | 7.0 | 8.4 |
| | 10 shampoos | 7.9 | 6.6 | 7.6 |
| shampoo 2BD4 | 0 | 7.2 | 6.7 | 7.4 |
| | 10 shampoos | 6.8 | 5.6 | 6.1 |
| shampoo 2BD4 + Serum | 0 | 12.9 | 10.7 | 8.3 |
| | 10 shampoos | 11.2 | 9.5 | 9.1 |
| shampoo 2BD5 | 0 | 5.3 | 3.9 | 4.4 |
| | 10 shampoos | 4.8 | 4.2 | 4.3 |
| shampoo 2BD5 + Serum | 0 | 11.7 | 7.7 | 8.7 |
| | 10 shampoos | 10.4 | 7.5 | 8.3 |

In the foregoing table the Volume Results were determined by ImageJ analysis of photomicrographs of hair fibers (software publicly available by NIH at https://imageJ.nih.gov). The treated hair fibers were found to have a range of volumes. A particularly effective synergistic result was observed with the initial combination of shampoos 2BD4 plus chitosan serum 4S and shampoo 2BD5 plus the chitosan serum 4s. For instance, the volume of the combination on virgin hair [12.9 cm for D4 (Lauryl PEG-10 Tris(Trimethylsiloxy) silylethyl Dimethicone), 11.7 cm for D5 (glycerin and water and polylysine)] was greater than the additive effect on volume produced by the serum and shampoo separately (5.5+7.2=12.7 for D4 or 5.5+5.3=10.8 for D5). The synergistic effect remained after 10 washes of the virgin hair for the dendrimer comprised of glycerin and water and polylysine (D5), i.e., the combination produced volume of 10.4 cm after 10 washes whereas the serum and shampoo separately only add to 10.1 cm (5.3+4.8).

A synergistic result was also observed after 10 washes of platinum bleached hair on which a combination of the hyperbranched amylopectin shampoo 2BD2 and serum 4S had been applied. That is, the volume of the hair after ten washes was 6.8 cm, which was higher than the additive effect of the shampoo and serum separately after 10 washes (2.0+4.3=6.3).

### Example 8: Color Retention

Color retention was evaluated for commercially purchased hair swatches that were dyed red with a commercially available PRAVANA permanent color kit. Red dyed hair was utilized for these experiments because red hair dye has been shown to lose color faster than other hair dyes. After the hair was dyed and blow-dried, the hair was shampooed with the various dendrimer shampoo compositions of Example 2, rinsed, treated with the chitosan complex serum of Example 4, and blow dried for 2 minutes with 10 passes on each side of the swatch. After treatment, consecutive stripping shampoos with blow-dries were performed.

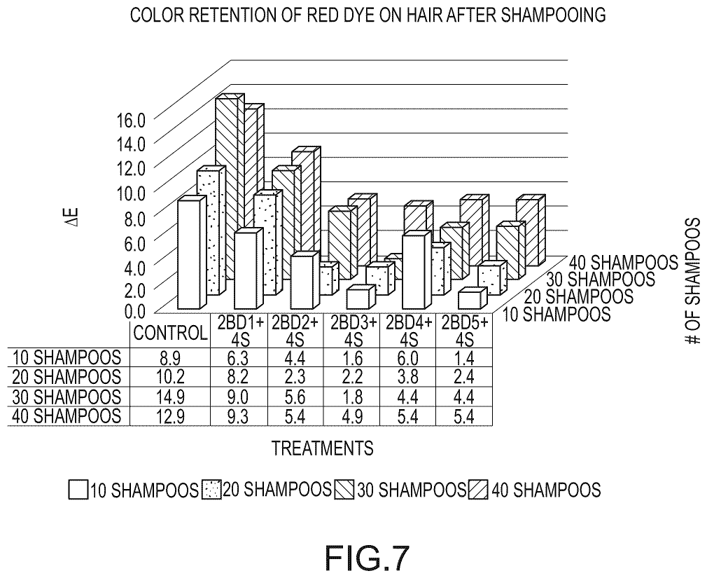

Hunter Color Coordinates (L A B values) were determined via laboratory measurement with a BYK color meter. FIG. 7 shows the color retention as measured by ΔE of the red color-treated hair swatches after 10, 20, 30, and 40 shampoos with Clarifying Shampoo. The ΔE was determined using ImageJ software publicly available by NIH at https://imageJ.nih.gov. The control was a dyed virgin hair swatch to which no Example 2 or Example 4 treatment was applied. After each stripping shampoo, the hair swatch was blow dried. An average of three measurements was used for the plotted ΔE values.

The color retention data indicates that the combination of the shampoos containing dendrimer or hyperbranched compound+chitosan serum helps lower the ΔE and improve the color retention for 40 shampoos with blow drying after each shampoo.

Overall, all evidence indicates that a combination hair treatment technology comprising a dendrimer primer composition and a chitosan complex composition provides long lasting color retention benefits. The different dendrimer shampoos do not alter the foaming properties of the shampoo and the chitosan complex with dehydroxanthan gum is a new complex that forms a stiff/sticky film with some flexibility. Without wishing to be bound by theory, it is believed the dendrimer/hyperbranched compound from the shampoo deposits on the hair, and traps the chitosan complex allowing it to stay on the hair for multiple washes.

What is claimed is:

1. A color retention system for color-treated hair comprising:
a primer composition comprising an effective amount of a dendrimer or hyperbranched compound in a cosmetically acceptable aqueous carrier; and
a chitosan complex composition comprising chitosan, dehydroxanthan gum, an aldehyde-bearing compound (ABC), and an organic acid
wherein upon application and rinsing of the primer followed by application of the chitosan complex composition, a composition is formed that provides color retention to the color-treated hair.

2. The color retention system of claim 1, wherein the dendrimer or hyperbranched compound is selected from the group consisting of polylysines, amylopectins, poly alpha-olefins, organopolysiloxane copolymers, polyquaterniums, and combinations thereof.

3. The color retention system of claim 1, wherein the dendrimer or hyperbranched compound is poly-L-lysine.

4. The color retention system of claim 1, wherein the dendrimer or hyperbranched compound is Hydrogenated Poly(C6-14 Olefin).

5. The color retention system of claim 1, wherein the dendrimer or hyperbranched compound comprises about 0.005 to about 5% by weight of the primer composition.

6. The color retention system of claim 1, wherein the primer composition is a shampoo that further comprises about 5 to about 30% by weight of active surfactant in a surfactant composition.

7. The color retention system of claim 6, wherein the surfactant composition comprises a surfactant selected from the group consisting of lauryl glucoside, caprylyl/capryl glucoside, cocoamidopropyl betaine, sodium lauroyl methyl isethionate, sodium methyl 2-sulfolaurate, sodium methyl cocoyl taurate, and combinations thereof.

8. The color retention system of claim 1, wherein the aldehyde-bearing compound (ABC) is selected from the group consisting of vanillin, p-vanillin, o-vanillin, or ethylvanillin.

9. The color retention system of claim 1, wherein the organic acid is selected from the group consisting of acetic acid, formic acid, glycolic acid, lactic acid, ascorbic acid, pyruvic acid, salicylic acid, citric acid, adipic acid, succinic acid, malic acid, tartaric acid, fumaric acid, glyoxilic acid, and combinations thereof.

10. The color retention system of claim 1, wherein the chitosan comprises about 0.3 to about 3% by weight of the chitosan complex composition.

11. The color retention system of claim 1, wherein the dehydroxanthan gum comprises about 0.1 to about 3% by weight of the chitosan complex composition.

12. A fiber color retention system comprising:
a primer composition comprising a dendrimer or hyperbranched compound; and
a chitosan complex composition comprising chitosan, an aldehyde-bearing compound, dehydroxanthan gum, and an organic acid
wherein upon application of the primer and the chitosan complex a composition is formed that provides color retention to the fiber.

13. The fiber color retention system of claim 12, wherein the primer composition comprises about 0.005 to about 5% by weight of the dendrimer or hyperbranched compound.

14. The fiber color retention system of claim 12, wherein the dendrimer or hyperbranched compound is selected from the group consisting of polylysines, amylopectins, poly alpha-olefins, organopolysiloxane copolymers, polyquaterniums, and combinations thereof.

15. The fiber color retention system of claim 12, wherein the dendrimer or hyperbranched compound is poly-L-lysine or Hydrogenated Poly(C6-14 Olefin).

16. The fiber color retention system of claim 12, wherein the aldehyde-bearing compound (ABC) is selected from the group consisting of vanillin, p-vanillin, o-vanillin, or ethylvanillin.

17. The fiber color retention system of claim 12, wherein the organic acid is selected from the group consisting of acid is selected from the group consisting of acetic acid, formic acid, glycolic acid, lactic acid, ascorbic acid, pyruvic acid, salicylic acid, citric acid, adipic acid, succinic acid, malic acid, tartaric acid, fumaric acid, glyoxilic acid, and combinations thereof.

18. The fiber color retention system of claim 12, wherein the chitosan comprises about 0.2 to about 3.0% by weight of the chitosan complex composition.

19. The fiber color retention system of claim 12, wherein the dehydroxanthan gum comprises about 0.1 to about 3% by weight of the chitosan complex composition.

20. A composition for use in retaining color on a fiber comprising a homogeneous dispersion of chitosan, dehydroxanthan gum, vanillin, and glacial acetic acid,
the chitosan comprising about 0.5 to about 1.0% by weight of the composition, and
the dehydroxanthan gum comprising about 0.25 to about 2.0% by weight of the composition
wherein the composition is free of brittle precipitates.

21. A method of maintaining initial color of color-treated hair comprising:
topically applying a shampoo composition comprising a dendrimer or hyperbranched compound to color-treated hair;
rinsing the shampoo from the hair;
topically applying an effective amount of a chitosan composition comprising a chitosan, dehydroxanthan gum, an aldehyde-bearing compound, and an organic acid to the rinsed hair; and
subsequently drying the hair so that the chitosan composition forms a crosslinked complex with the hair.

22. The method of claim 21, wherein the crosslinked complex is maintained on the hair for 10 washes with a clarifying shampoo.

23. The method of claim 21, wherein the shampoo is free of sulfates.

24. The method of claim 21, wherein the chitosan composition is free of precipitates.

25. The method of claim 21, wherein the step of drying comprises blowing the hair with air at temperature of about 50° C. or greater for about 2 minutes.

26. The method of claim 21, wherein the ΔE of the dried hair is less than or equal to 10 after 40 washes with a clarifying shampoo.
